(19) United States
(12) Patent Application Publication
(10) Pub. No.: US 2010/0266727 A1
(43) Pub. Date: Oct. 21, 2010

(54) STABILISATION OF DRIED BIOLOGICAL MATERIAL

(75) Inventors: Jayanthi Swaminathan, Christchurch (NZ); Trevor Anthony Jackson, Christchurch (NZ)

Correspondence Address: SUGHRUE MION, PLLC, 2100 PENNSYLVANIA AVENUE, N.W., SUITE 800, WASHINGTON, DC, 20037, US

(73) Assignee: ENCOATE HOLDINGS LIMITED, Hamilton (NZ)

(21) Appl. No.: 12/741,791

(22) PCT Filed: Nov. 6, 2008
(86) PCT No.: PCT/NZ2008/000300

§ 371 Date: May 6, 2010

(30) Foreign Application Priority Data
Nov. 7, 2007 (NZ) 555022

(51) Int. Cl. A23L 1/30; C12N 1/00; C12N 1/20; C12N 1/14

(52) U.S. Cl. 426/61; 435/243; 435/252.1; 435/252.9; 435/253.4; 435/254.1

(57) ABSTRACT

The invention relates to a storage stable composition and method of manufacture containing a biological material, oil and a biopolymer which has a water activity of less than 0.5. The biological material in the composition attains significant stability and resistance to temperature, humidity and oxygen degradation. Advantages from the invention include the ability to stabilise, further process, store and then release the probiotic bacteria at a later date and retain viability. In addition the composition and method is more practical for producing and marketing of biological materials including probiotic material as it is simple, has low energy requirements and the resulting product is easy to incorporate in formulations and foods.

### STATEMENT OF CORRESPONDING APPLICATIONS

This application is based on the Provisional specification filed in relation to New Zealand Patent Application Number 555022, the entire contents of which are incorporated herein by reference.

### TECHNICAL FIELD

The invention relates to stabilisation of dried biological material. More specifically, the invention relates to a composition containing a biological material and method for manufacturing the composition where the biological material is storage stable.

### BACKGROUND ART

Biological materials tend to be unstable when stored outside of preferred conditions in terms of heat, humidity moisture and so forth. Stabilising such materials is of key importance when manufacturing goods for later use such as in foods, agricultural products, laboratory materials and so on.

The following discussion describes the stability issue in terms of probiotic bacteria although it should be appreciated that the issue is not specific to such bacteria but rather that, the example of probiotic illustrates one part of the problem to be solved.

Probiotics are considered to be viable microbial preparations which promote mammalian health by preserving the natural microflora in the intestine. Probiotics are thought to attach to the intestinal mucosa, colonize the intestinal tract and thereby prevent attachment of harmful micro-organisms thereon. A prerequisite for their action resides in that they have to reach the gut's mucosa in a proper and viable form and especially do not get destroyed by the low pH in the stomach.

A known problem associated with delivery of active biological material, particularly in foods, is the maintenance of the materials in a viable state or a stable state until they are used. Viability also needs to continue through the stomach so that the probiotic delivers activity to the gut. Finally, storage stability is also important as many biological materials cannot be maintained in a viable condition during long term storage, particularly when stored at ambient conditions.

At present, bacterial biological materials require production of high concentrations of bacteria to ensure survival of commercially useful numbers for extended periods. This has been achieved to a limited degree using refrigeration and/or freeze drying to preserve viability. Additionally, while some microbial products require only the delivery of an inoculative dose, for others including probiotics, delivery of a higher minimum dosage concentration is essential to the success of the product.

The inventors found that besides normal issues with stability, probiotic bacteria in a dried state have additional problems in that they are particularly sensitive to degradation in ambient conditions. This is due to the highly hydroscopic nature of the dried bacterium which results in the bacteria rapidly loosing viability in humid and/or warm environments.

To illustrate the importance of stabilisation for such applications, reference is made to WO 01/90311 (Nestle) which describes pet foods incorporating isolated strains of probiotic bacteria. The bacteria need to be able to produce at least 1×10⁶ cfu/ml after about 2 hours at a pH range from about 3.4 to about 4.2 (stomach bile acid conditions). In the '311 publication, no specifics are provided on the method of stabilisation, rather the bacteria are selected for their properties. The inventor's experience is that, without some form of stabilisation the probiotic bacteria will rapidly lose viability below the 1×10⁶ cfu/ml level, particularly when stored at room temperature prior to use.

Another patent application that also teaches the importance of delivering probiotics viably is WO 2005/060709 (Proctor and Gamble). The '709 application describes compositions including a probiotic strain of Bifidobacteria obtained by isolation from resected and washed mammalian gastrointestinal tract. The '709 publication recognises the importance of stabilising the bacteria as it is noted that the Bifidobacteria need to be able to maintain viability following transit through the GI tract. This is desirable in order for live cultures of the bacteria to be taken orally, and for colonisation to occur in the intestines and bowel following transit through the oesophagus and stomach. It is noted that oral dosing of non-viable cells or purified isolates thereof induces temporary benefits, but as the bacteria are not viable, they are not able to grow, and continuously deliver a probiotic effect in situ. The '709 publication describes that it is preferable that the lactic acid bacteria of the present invention maintain viability after suspension in a media having a pH of 2.5 for 1 hour (equivalent to stomach acid digestion). Little specifics are taught regarding stabilisation methods other than careful processing e.g. nitrogen flushing and use of sealed containers during processing and for storage. Delivery methods are only described as including the probiotic within dried animal foods such as biscuits or kibbles, processed grain feed, wet animal food, yogurts, gravies, chews, treats and the like.

One method that has tried to address the stability issue is that described in WO2005/030229 (Crittendon). In the '229 publication, Bifidobacterium infantis probiotic strain is microencapsulated within a film-forming, protein-carbohydrate-oil emulsion. The encapsulation step is said to protect the bacterium during non-refrigerated storage and gastrointestinal transit. The microcapsules are described as being small (15 to 20 μm), with low water activity (0.2 to 0.3), and rapidly release the bacteria in simulated intestinal fluid.

The shelf life of the dried microcapsules produced using the method of the '229 publication under non-refrigerated storage (25° C. at 50% relative humidity) was significantly higher than that of non-encapsulated bacteria. The '229 publication teaches of a loss of approximately 2 logs over 5 weeks which, in the inventors experience, is still not ideal.

This loss is understood by the inventors to be attributable to the microbial material being suspended in an aqueous solution (re-hydrated) and then spray dried at high temperatures. Re-hydration and exposure to an aqueous environment is undesirable as this makes the bacteria highly prone to degradation. In addition spray drying is energy intensive hence has a proportional manufacturing cost. Further, spray drying can expose the bacteria to elevated temperatures whilst the bacteria is not fully stabilised which may also lead to a loss in viability.

Prior art described in the '229 publication outlines further ways to protect the probiotic bacteria including: encapsulation in a slow release pharmaceutical compound; encapsulation in a gum or in alginate; encapsulation in a resistant starch in combination with a gum; protection by incorporation in a food containing resistant starch; or in a dairy food where the proteins and fats may provide some protection.

One example of the above includes the applicant's previous patent application published as WO 02/15702, incorporated herein by reference. WO02/15702 describes a method of producing a stable bio-matrix gel by use of a biopolymer gum. Whilst this is useful in providing a stabilised agent, the raw material is primarily a liquid biological, and a gel is not always the preferred delivery mechanism as it still has an aqueous component.

Some probiotics need protection during processing as well as during delivery to the gastro intestinal tract. Some probiotics may be water or oxygen sensitive and need protection to maintain viability during processing, storage and transportation. For example, European patent 1213347 discloses a method of drying and preserving yeasts and micro-organisms by mixing them with a matrix material that absorbs water

Other examples of prior art methods include:

U.S. Pat. No. 5,422,121 discloses a coating incorporating a film forming polymer having hydrophilic groups and a polysaccharide which is decomposable in the colon. Such coatings are useful in delivering dosages to the colon.
U.S. Pat. No. 5,840,860 discloses the delivery of short chain fatty acids to the colon by covalently linking them to a carbohydrate desiccant.
U.S. Pat. No. 6,060,050 discloses a combination of probiotic bacteria such as Bifidobacteria with high amylose starch as a desiccant which also acts as a growth or maintenance medium in the large bowel or other regions of the gastrointestinal tract (GI tract).
USA patent application 2003/0096002 discloses a matrix for use in the controlled release of micro-organisms. The matrix is formed of a hydrophobic wax and a release modifying agent selected from polysaccharides, starch, an algae derivative or a polymer.
U.S. Pat. No. 6,413,494 discloses a colonic drug delivery vehicle consisting of a polysaccharide such as pectin.

As may be noted from general literature regarding probiotic bacteria, there are a large number of emerging health claims made for probiotics. These centre particularly on bowel action and include treatments for bowel cancer, irritable bowel syndrome and inflammatory bowel diseases (such as Crohn's disease). Given the importance of these conditions, preparation and delivery of stabilised probiotic agents is a critical step. In particular, for probiotic bacteria, the method must be able to address the extra sensitivity of such bacteria as well as maintaining viability of the bacteria viable through the stomach and into the GI tract.

As should also be appreciated from the above discussion, the problems faced in stabilising probiotic bacteria also occur for other types of biological materials and that therefore compositions and methods addressing probiotics may also be useful for other biological materials.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF THE INVENTION

According to one aspect of the present invention there is provided a storage stable composition containing a biological material, oil and a biopolymer and,
wherein the composition has a water activity of less than 0.7 and,
wherein the biological material in the composition remains viable when the composition is stored at ambient temperatures.

According to a further aspect of the present invention there is provided a method of producing a storage stable composition including a stabilised biological material by the steps of:

(a) obtaining a dried biological material;
(b) coating the dried biological material with at least one oil to form a oil coated material;
(c) coating the oil coated material with at least one biopolymer substance to produce the composition; and,
wherein the biological material in the composition remains viable when the composition is stored at ambient temperatures.

According to a further aspect of the present invention there is provided a food including a storage stable composition substantially as described above.

Preferably, the food is substantially dry and stored at ambient temperature and humidity.

According to a further aspect of the present invention there is provided a nutraceutical product including a storage stable composition substantially as described above.

According to a further aspect of the present invention there is provided a food ingredient including a storage stable composition substantially as described above.

According to a further aspect of the present invention there is provided a bran flake coated in the composition substantially as described above.

According to a further aspect of the present invention there is provided a milk powder including the composition substantially as described above.

According to a further aspect of the present invention there is provided an infant formula including the composition substantially as described above.

According to a further aspect of the present invention there is provided an animal food including the composition substantially as described above.

Preferably, the term ‘stable’ or grammatical variations thereof refers to the biological material retaining sufficient viability to be commercially useful during processing and storage. More specifically, stability may be defined as being:
(a) a biological viability of less than 2 log loss in viability when stored in a sealed environment for at least 1 month at 25° C. to 30° C.; and/or,
(b) biological material having less than 50% loss in viability when stored in unsealed or sealed environments at 60% relative humidity and at a temperature 25° C. to 30° C. for a time period of at least 3 days.

More preferably, the biological viability may be less than 1 log loss in viability when stored in a sealed environment for at least one month at 25° C. to 30° C.

As may be appreciated, the above stability may be of great advantage in processing as it avoids the need to conduct processing in special conditions. The long term storage stability may also be useful to allow for product transport and sale.

Preferably, the term ‘ambient’ refers to normal room temperatures, humidity's and atmospheric pressure. More specifically this term refers to a temperature ranging from approximately 10° C. to 50° C., more preferably 15 to 25° C., and a relative humidity ranging from 0% to 70%, more preferably 40-80% and standard atmospheric pressure.

Preferably, the method substantially as described above may be conducted at ambient conditions. In addition, it may not be necessary to use oxygen free environments during or after processing, although this may optionally be done.

A key aspect in development of the above composition and method was the recognition that aqueous materials and environments were to be avoided throughout the process, and that it was critical to reduce the water activity significantly without subjecting the biological material to elevated temperatures or re-hydrating the biological material. The solution arrived at by the inventors was to modify the biological material using other components to give a less hydrophilic (and more hydrophobic) end material.

Increased hydrophobicity may be highly advantageous in making the composition more stable. Humidity is a key problem in processing and handling dried biological materials. Some materials such as obligate anaerobes, including probiotic bacteria, are very sensitive to humidity and oxygen even once dried. By changing the nature of the composition, a resistance has been developed to humidity and oxygen which allows processing to be completed in conditions that would not normally be possible when processing such materials, due to unacceptable losses in viability. For example, prior art teaches use of nitrogen flushing and sealed containers during processing which is not a requirement of the present invention.

Preferably, the dried biological material of step (a) may have a water activity of less than 0.7. More preferably, the water activity may be less than 0.4. Still more preferably, the water activity may be less than 0.2. It should be appreciated that this is a low water activity and as a result, the biological material is stabilised due a least in part to the low water activity environment of the composition.

Preferably, the dried biological material may be pre-processed by freeze drying or lyophilisation. Alternatively, other drying processes may have been used prior to stabilisation in the present invention, for example spray or air drying. Although spray dried and air dried biological materials have no impact on the stabilisation method of the present invention, these methods are less desirable as they may reduce viability prior to stabilisation in this method at a greater rate than other methods such as freeze drying.

Preferably, the dried biological material may be a powder. Preferably, the powder may have a particle size of less than approximately 2 mm, more preferably, less than 200 μm. Smaller sized particles may be preferable although not essential as this allows for better mixing and homogeneity.

Preferably, the biological material may contain at least one micro-organism.

Preferably the biological material may be bioactive such that it may have an interaction with cell tissue.

In preferred embodiments, the biological material may contain at least one bacteria or fungi including yeasts.

In one embodiment the biological material may contain gram negative bacteria. Gram negative bacteria may be selected from the genus: Serratia, Pseudomonas, Xanthamonas, Rhizobium, and combinations thereof.

In an alternative embodiment the biological material may contain gram positive bacteria. Gram positive bacteria may include probiotic bacteria and Staphylococcus genus bacteria.

In a further embodiment, the biological material may contain obligate anaerobic bacteria Obligate anaerobic bacteria may include probiotic bacteria and Bacteroides genus bacteria.

In a particularly preferred embodiment the biological material may contain probiotic bacteria or fungi. For the purposes of this specification, the term ‘probiotic’ refers to viable bacteria and fungi such as yeasts that beneficially influence the health of the host.

Probiotic bacteria include those belonging to the genera Lactococcus, Streptococcus, Pediococcus, Enterococcus, Leuconostoc, Carnobacterium, Propionibacterium, Lactobacillus or Bifidobacterium.

Bifidobacteria used as probiotics include Bifidobacterium adolescentis, Bifidobacterium bifidum, Bifidobacterium animalis, Bifidobacterium thermophilum, Bifidobacterium breve, Bifidobacterium longum, Bifidobacterium infantis and Bifidobacterium lactis. Specific strains of Bifidobacteria used as probiotics include Bifidobacterium breve strain Yakult, Bifidobacterium breve R070, Bifidobacterium lactis Bb12, Bifidobacterium longum R023, Bifidobacterium bifidum R071, Bifidobacterium infantis R033, Bifidobacterium longum BB536 and Bifidobacterium longum SBT-2928.

Lactobacilli used as probiotics include Lactobacillus acidophilus, Lactobacillus brevis, Lactobacillus bulgaricus, Lactobacillus casei, Lactobacillus cellobiosus, Lactobacillus crispatus, Lactobacillus curvatus, Lactobacillus fermentum, Lactobacillus GG (Lactobacillus rhamnosus or Lactobacillus casei subspecies rhamnosus), Lactobacillus gasseri, Lactobacillus johnsonii, Lactobacillus plantarum and Lactobacillus salivarus. Lactobacillus plantarum 299v strain originates from sour dough. Lactobacillus plantarum itself is of human origin. Other probiotic strains of Lactobacillus are Lactobacillus acidophilus BG2FO4, Lactobacillus acidophilus INT-9, Lactobacillus plantarum ST31, Lactobacillus reuteri, Lactobacillus johnsonii LA1, Lactobacillus acidophilus NCFB 1748, Lactobacillus casei Shirota, Lactobacillus acidophilus NCFM, Lactobacillus acidophilus DDS-1, Lactobacillus delbrueckii subspecies delbrueckii, Lactobacillus delbrueckii subspecies bulgaricus type 2038, Lactobacillus acidophilus SBT-2062, Lactobacillus brevis, Lactobacillus salivarius UCC 118 and Lactobacillus paracasei subsp paracasei F19.

Lactococci that are used or are being developed as probiotics include Lactococcus lactis, Lactococcus lactis subspecies cremoris (Streptococcus cremoris), Lactococcus lactis subspecies lactis NCDO 712, Lactococcus lactis subspecies lactis NIAI 527, Lactococcus lactis subspecies lactis NIAI 1061, Lactococcus lactis subspecies lactis biovar diacetylactis NIAI 8W and Lactococcus lactis subspecies lactis biovar diacetylactis ATCC 13675.

Streptococcus thermophilus is a gram-positive facultative anaerobe. It is a cytochrome-, oxidase- and catalase-negative organism that is nonmotile, non-spore forming and homofermentative. Streptococcus thermophilus is an alpha-hemolytic species of the viridans group. It is also classified as a lactic acid bacteria (LAB). Streptococcus thermophilus is found in milk and milk products. It is a probiotic and used in the production of yogurt. Streptococcus salivarus subspecies thermophilus type 1131 is a probiotic strain.

Enterococci are gram-positive, facultative anaerobic cocci of the Streptococcaceae family. They are spherical to ovoid and occur in pairs or short chains. Enterococci are catalase-negative, non-spore forming and usually nonmotile. Enterococci are part of the intestinal microflora of humans and animals. Enterococcus faecium SF68 is a probiotic strain that has been used in the management of diarrhoeal illnesses.

The principal probiotic yeast may be Saccharomyces boulardii. Saccharomyces boulardii is also known as Saccharomyces cerevisiae Hansen CBS 5296 and S. boulardii. S. boulardii is normally a non-pathogenic yeast. S. boulardii has been used to treat diarrhoea associated with antibiotic use.

Preferably, the initial cell concentration of the bacteria or fungi in the dried raw material may be in the range of 10⁵ cells to 10¹² cells per gram, more preferably, between 10⁷ and 10¹⁰ cells per gram.

Preferably, the oil of step (b) may be an edible oil. In one embodiment, the oils may be marine oils including but not limited to fish oils and algal oils. In another embodiment, the oil may be a vegetable oil. In preferred embodiments, the vegetable oil may be selected from: olive oil, canola oil, sunflower seed oil, hydrolyzed oils, and combinations thereof. The above oils should not be seen as limiting as it should be appreciated that other oils with similar chemical and physical characteristics may be used without departing from the scope of the invention. It is understood by the inventors that oil may be useful to help protect the biological material from moisture and assists in changing the hydrophilic property of the dried biological material to a hydrophobic property, also assisting in stabilising the biological material.

In one preferred embodiment, the oil used may have high levels of antioxidants, such as but not limited to, cold pressed virgin oils. It is understood by the inventors that use of oil with high levels of antioxidants may assist in preventing a loss in viability due to oxidative degradation when the biological material is exposed to the environment.

Preferably, sufficient oil may be added to fully coat the dried biological material. In one preferred embodiment, the ratio of dried biological material to oil may be in the range 1:10 to 10:1 by weight. In a more preferred embodiment, the ratio of dried biological material to oil may be from 1:1 to 1:4. In a yet more preferred embodiment the ratio may be approximately 1:2.

Preferably, the biopolymer material of step (c) may be a natural or synthetic gum.

Preferably, the biopolymer gum used may be characterised by having a molecular weight of between 5000 and 50 million. The biopolymer gum may also be characterised by being resistant to enzymatic degradation as well as being resistant to shear, heat, and UV degradation. In preferred embodiments, the gum when mixed in the composition may also confer pseudoplastic properties to gels produced.

In one preferred embodiment, the biopolymer may be a gum selected from agar, alginate, cassia, dammar, pectin, beta-glucan, glucomannan, mastic, chicle, psyllium, spruce, gellan, guar, locust bean, xanthan, and combinations thereof. It should be appreciated that the above gums are provided by way of example and that other gums with equivalent chemical and physical properties may also be used, such as those with equivalent swelling and moisture absorption characteristics.

Preferably, the biopolymer used may be in powder form. Preferably, the powder may have a particle size of less than 2 mm.

Preferably, the biopolymer may be added a rate of 1 part biological material to between 0.25 to 1 parts biopolymer. More preferably, the ratio may be 1 part biological material to ⅔^{rd} or 0.66 parts biopolymer.

In a further embodiment, at least one antioxidant may be added in addition to any antioxidant oils. In one embodiment, the antioxidant may be mixed tocopherol (vitamin E).

Preferably, antioxidant may be added at a ratio of 1 gram dried biological material to 5-1004 of mixed tocopherol antioxidant prepared according to manufacturer's specifications. In a preferred embodiment the ratio may be 1 gram dried biological material to 40 μL of mixed tocopherol antioxidant.

Preferably, antioxidant is added is added after coating with oil, but before addition of biopolymer.

It should be appreciated that use of antioxidants such as oils rich in antioxidants as well as other compounds such as vitamin E may be advantageous as it helps to prevent oxidation. Probiotic bacteria thrive in the anaerobic environment of the gastrointestinal tract and therefore deteriorate quickly in aerobic environments such as in storage. Methods to deal with this normally involve storage in vacuum sealed environments, only storing material for limited time periods and avoiding oxygen exposure as much as possible. By stabilising the probiotic bacteria using a method that introduces antioxidant containing compounds, the resulting composition exhibits a resistance to oxidation deterioration of the biological material.

In a yet further embodiment, the composition may be further formulated by adding at least one desiccant substance. For the purposes of this specification, the term ‘desiccant’ will be used and encompasses materials that are largely dry and chemically inert powders with respect to the composition. In embodiments developed by the inventors, desiccant substances may be selected from: rice powder, corn starch powder, potato starch powder, lactose, sucrose, glucose, mannitol, sorbitol, calcium carbonate, silicone dioxide, calcium phosphate, celluloses, polyethylene glycol, and combinations thereof. It should be appreciated by those skilled in the art that the above list is provided by way of example and that desiccants of the art in general may be added depending on the end application e.g. food applications require food safe desiccant substances.

Preferably, where added, the desiccant substances may be included at a rate of 1 part composition from step (c) to between 0.5 and 4 parts desiccant. More preferably, the ratio may be 1 part composition from step (c) to 1 part desiccant.

Preferably, the desiccants where used may be pre-dried to reduce the water activity to less than 0.4, more preferably to less than 0.1. In one embodiment, this may be achieved by air drying for approximately 4 to 18 hours. This should not be seen as limiting as other drying methods may be used without departing from the scope of the present invention. Air drying may be preferable though due to its simple process requirements, minimal preparation time and the fact that it can be operated to avoid the need for temperatures in excess of 40° C. It should be appreciated that higher temperatures than 40° C. may be used without departing from the scope of the invention.

An advantage the inventors have found is that desiccant substances may assist in further reducing the composition water activity. In the inventor's experience, water activity in the final composition may be reduced to as low as approximately 0.04 to 0.08 by use of desiccants.

Preferably, the desiccants when used are added in multiple stages. In one example, desiccant may be added at a rate of 1 to 10 gram additive per stage per 1 gram of dried cells used. Preferably, a total of up to 20 grams of desiccants may be used per 1 gram of dried cells.

In one embodiment, the amount of desiccant used may be based on what final product consistency is desired in the composition. For example, in the inventor's experience a rough crumble results from addition of a small amount of desiccant and, as more desiccant is added, the composition becomes more powder like.

It should be appreciated that the composition produced may be extruded or pressed into tablets, granules, prills or pellets. The inventors have found that in one example, the addition of the first 20 or 40 grams of additive is sufficient to produce a good consistency for extrusion.

In a further embodiment, the formulation may be applied to a substrate for example, by coating the substrate. By way of example, the formulation may be coated onto a bran flake for use in cereal applications.

Preferably, the composition produced may be stored in a sealed environment. By way of example, the composition may be stored in bags or sealed polystyrene containers. This is to help protect the composition from attack by humidity or oxidative degradation.

An advantage found by the inventors is that the composition may not need to be vacuum sealed. Unlike prior art methods, removal of oxygen from a container prior to sealing may not be essential and has appears to have a negligible effect on viability.

A further advantage of the above method is that the process may not need to be completed under special temperature, humidity or inert atmospheres unlike prior art methods. By way of example the inventors have found good process efficiencies where the efficiency is a percentage measure between levels of viable cells before and after processing. In the inventor's work the difference in cell count before and after processing is normally no more than 2 log loss, more preferably, less than 1 log loss. In the inventor's experience, the loss is typically far less with measured efficiencies of over 80% for at least Bifidobacterium genus bacteria.

Another factor addressed in the present invention may be oxidation and prevention of this occurring. Prevention of oxidation may be useful as it avoids the need for special packaging e.g. there is no requirement for vacuum or nitrogen flushing of packaging.

A further advantage of the present invention is that the composition may still be easily rehydrated for use in applications such as tablets or capsules for oral administration. In addition, due to the substances used being food safe, the stabilised formulation may also be added or coated onto foods such as crackers, breads, dried pet foods and the like, and then packaged and sold for later use as required.

Importantly, the viability also did not decrease significantly before and after processing which is an improvement on existing methods such as that of Crittendon, which utilises aqueous materials and exhibit a significant drop in viability during processing.

Based on the inventor's work in this area, the probiotic bacteria is envisaged as being stable for over 12 months without the cell concentration reducing below 2 log loss of original levels.

It should be appreciated from the above description that there is provided a method and composition that offers considerable advantages over the prior art including:
The ability to stabilise, further process, store and then release the probiotic bacteria at a later date and retain viability;
Hydrophilic properties being converted to hydrophobic properties;
A very low residual water activity and hence high stability environment;
Protection from oxidation reactions; and,
A more practical method for producing and marketing of probiotic material as the method is simple, has low energy requirements and the resulting composition is easy to incorporate in formulations and foods.

### BRIEF DESCRIPTION OF THE DRAWINGS

Further aspects of the present invention will become apparent from the following description, which is given by way of example only and with reference to the accompanying drawings in which:

FIG. 1 shows a graph indicating the cell survival (%) for a formulated or unformulated Lactobacillus acidophilus when stored at different temperatures and relative humidity's in closed packaging;

FIG. 2 shows a graph indicating the cell survival (%) for a formulated or unformulated Lactobacillus acidophilus when stored at different temperatures and relative humidity's in open packaging;

FIG. 3 shows a graph of the cell survival (%) for a formulated (closed symbol) or unformulated (open symbol) Bifidobacterium lactis when stored at 25° C.;

FIG. 4 shows a graph of the cell survival (%) for a formulated (closed symbol) or unformulated (open symbol) Bifidobacterium lactis when stored at 30° C.;

FIG. 5 shows a graph of the cell survival (%) for a formulated (closed symbol) or unformulated (open symbol) Lactobacillus GG when stored at 25° C.;

FIG. 6 shows a graph of the cell survival (%) for a formulated (closed symbol) or unformulated (open symbol) Lactobacillus GG when stored at 30° C.; and,

FIG. 7 shows a graph illustrating the survival (%) at 30° C. for a Lactobacillus containing formulation over 2 months where the first bar refers to the % survival after one month and the second bar refers to the % survival after 2 months. Formulations are labelled as follows: ‘ll6’ refers to a formulation containing 2 parts oil and 0.34 parts biopolymer to freeze dried bacteria; ‘lh6 ’ refers to a formulation containing 2 parts oil and 0.68 parts biopolymer; ‘hl6 ’ refers to a formulation containing 3 parts oil and 0.34 parts biopolymer; ‘hh6’ refers to a formulation containing 3 parts oil and 0.68 parts biopolymer.

### BEST MODES FOR CARRYING OUT THE INVENTION

Examples are now described for the method of stabilising various dried biological materials and results shown of the stability observed.

### Example 1

In this example, a general method of producing stabilised biological material is described.

The method involves the steps of:

1. Mixing oil with dried biological material (e.g. cells). For this example, the oil is cold pressed extra virgin olive oil added in the ratio of 1 part cells to 2 parts oil. An aim of this step is to thoroughly coat the dried cell particles with oil. Olive oil is used although other oils such as other vegetable oils may also be used.
2. Optionally, an antioxidant may be added to the mixture of step 1 e.g. Prepared Vitamin E/Mixed Tocopherol. It is understood that addition of further antioxidant is not essential but may be advantageous. Mixed tocopherol is prepared as per manufacturer's recommendations.
3. A biopolymer material is then added. In this example, gums such as gellan, or a combination of gellan and guar gum is used. Other gums may also be used including xanthan, locust bean and others with similar properties. The gum is added at an approximate ratio of 1 gram of dried cells to 0.66 gram of biopolymer. The gum is in solid particulate form.

In the above method, the formulations are prepared ideally under aseptic conditions at ambient temperatures, with the relative humidity being at standard laboratory conditions of approximately 50%. It should be noted that no special handling conditions are required beyond that described above, unlike prior art methods which may require chilling and low humidity conditions.

The resulting product is stable at ambient temperature and may be packaged in sealed bags although vacuum sealing is not essential.

The formulation may also be mixed with other materials to form final products such as baby formula or may be coated on substrates e.g. bran flakes in the manufacture of a breakfast cereal.

### Example 2

The above method described in Example 1 may also include an optional further step of adding a desiccant or desiccants.

Desiccants including starch (corn starch), rice powder, Paselli BC (potato starch) or combinations of these desiccants may also be added at a ratio of 1 part additives to 1 part mixture of step 3. It should be appreciated from the above description of the invention, that addition of desiccants is not essential to the method.

In this example, the desiccant(s) are prepared before addition to the agent mixture of step 3 by oven drying overnight at a temperature of approximately 80° C. or less, and then cooling to ambient temperature prior to being used in manufacturing the formulation. The aim of this preparation step is to reduce the water activity of the desiccant(s) to less than approximately 0.1.

Desiccant(s) are added to the bulk in a series of four separate stages to reduce the water activity of the powder progressively. Desiccant(s) are added at a rate of 1 to 10 (preferably 5 gram lots) grams additive per stage per 1 gram of dried cells used. In total, up to 20 grams of desiccant(s) may be used per 1 gram of dried cells.

During each stage, the mixture transforms from a rough crumble after stage 1 through to a fine powder texture or crumble after the fourth stage.

As noted in the above description of the invention, it is not essential to use desiccant(s), nor is it essential to add all four stages of desiccant. For example, the mixture could also be extruded into tablets, granules, prills or pellets as well, especially after the addition of the first 20 or 40 grams of additive.

For the purposes of this example, the total proportions by weight of the different ingredients used are as shown in Table 1:

TABLE 1
Approximate Composition of Final Formulation

| | Amount |
|---|---|
| Component | (% wt) |
| Dried cells: | 4.3% |
| Oil | 8.7% |
| Antioxidant (if added) | 0.2% |
| Desiccant(s) | 86.8% |

### Example 3

In this trial, three different formulations using Bifidobacterium infantis strain were stabilised and tested for storage stability when stored in vacuum packed and sealed aluminium foil bags.

The raw material used was a freeze dried powder containing B. infantis cells obtained from a commercial supplier and received in sealed foil packages stored at −20° C. The freeze dried powder had a water activity of approximately 0.4 and an initial cell count of approximately 1.2×10¹¹ cfu/g.

Three different formulations were prepared as follows:

### Formulation 1 (F1):

(a) 1 gram of freeze dried powder was mixed with 2 grams of extra virgin olive oil;
(b) 40 μl of prepared antioxidant (mixed tocopherol) was added;
(c) 0.33 grams biopolymer (gellan gum) was added;
(d) 20 grams of additive being oven dried rice powder (Paselli BC (1:1)) was then added to the bulk in 5 gram increments where the rice powder before addition had a water activity (A_{W}) of 0.024.

### Formulation 2 (F2):

(a) 1 gram of freeze dried powder was mixed with 2 grams of salad and cooking oil;
(b) 40 μl of prepared antioxidant (mixed tocopherol) was added;
(c) 0.17 grams gellan gum and 0.17 grams guar gum was added being the biopolymer;
(d) 20 grams of additive being oven dried rice powder (Paselli BC (1:1)) was then added to the bulk in 5 gram increments where the rice powder before addition had a water activity (A_{W}) of 0.024.

### Formulation 3 (F3):

(a) 1 gram of freeze dried powder was mixed with 2 grams of salad and cooking oil;
(b) 40 μl of prepared antioxidant (mixed tocopherol) was added;
(c) 0.17 grams gellan gum and 0.17 grams guar gum was added being the biopolymer;
(d) 20 grams of additive being potato starch was then added to the bulk in 5 gram increments where the potato starch before addition had a water activity (A_{W}) of 0.031.

The resulting mixture had a fine crumble texture.

5 gram samples from each formulation were then placed in vacuum packaged foil sachets and stored at 25° C. to test stability over time. When tested, the sachets were destructively sampled and bacterial density enumerated by anaerobic culture on Reinforced Clostridial Agar (RCA) as well as water activity. Water activity was also measured for the formulation before storage.

### Results

As shown in Table 2 below, the water activity after mixing and after storage showed little change. Water activity in both cases was extremely low (all less than 0.1). As may be noted, the water activity in fact decreased over time. This is thought to be because over time, any remaining free water is taken up by the formulation ingredients.

TABLE 2
Measured Sample Water Activity - F1, F2, F3

| | | Tested Water Activity |
|---|---|---|
| Formulation | Tested Water Activity | After Storage (t = 2 |
| Number | After Mixing (t = 0) | months) |
| F1 | 0.06 | 0.03 |
| F2 | 0.082 | 0.032 |
| F3 | 0.073 | 0.053 |

Stability results shown below in Table 3 showed that there was a less than 1 log loss in bacterial cell counts over a time period of at least 2 months. The process efficiency was also excellent as, using the average count after mixing, 89% of the initial cell count was still present after formulating.

TABLE 3
Measured Stability - 2 months - B. infantis - F1, F2, F3

| | Bacterial | | Bacterial |
|---|---|---|---|
| | Count After | Bacterial | Count After 2 |
| Formulation | Mixing (t = 0) | Count After 1 | months |
| Number | (cfu/g) | month (cfu/g) | (cfu/g) |
| 1 | 4.81E+09 | 3.08E+09 | 4.27E+09 |
| 2 | 1.38E+09 | 3.83E+09 | 2.94E+09 |
| 3 | 3.42E+09 | 6.74E+09 | 2.71E+09 |

The above trial shows that different biopolymers may be used in the above method without altering the improved stabilisation results. In addition, the above trial shows that different oils and additives may also be used without altering the stabilisation results.

### Example 4

In this trial, a different formulation was used to stabilise a B. infantis strain with the aim of testing the stabilisation result over a longer duration (6 month) time period. Again, the freeze dried powder had a water activity of approximately 0.4.

The formulation was made as follows:

### Formulation 4 (F4):

(a) 1 gram of freeze dried powder was mixed with 2 grams of extra virgin olive oil;
(b) 40 μl of prepared antioxidant (mixed tocopherol) was added;
(c) 0.17 grams gellan gum and 0.17 grams guar gum was added being the biopolymer;
(d) 20 grams of oven dried rice powder (Paselli BC (1:1)) was then added to the bulk in 5 gram increments where the rice powder before addition had a water activity (A_{W}) of 0.024.

The resulting mixture had a fine crumble texture.

10 gram samples were then placed in vacuum packaged foil sachets and stored at 25° C. to test stability over time. When tested, the sachets were destructively sampled and bacterial density enumerated by anaerobic culture on Reinforced Clostridial Agar (RCA).

### Results

The results found as shown in Table 4 below showed that the method stabilised the bacteria sufficient that over a 6 month time period, the bacterial cell count remained within 1 log of the original (t=0) bacterial cell count.

TABLE 4
Measured Stability - 6 months - B. infantis - F4

| | Bacterial cell counts | Bacterial cell counts | Bacterial cell counts | Bacterial cell counts | Bacterial cell counts |
|---|---|---|---|---|---|
| | after mixing t = 0 | after 1 month | after 2 months | after 4 months | after 6 months |
| Formulation | (cfu/g) | (cfu/g) | (cfu/g) | (cfu/g) | (cfu/g) |
| F4 | 3.80E+09 | 2.87E+09 | 4.43E+09 | 5.09E+09 | 3.00E+09 |

The above Example shows that further modifications to the method may be made without altering the stabilisation result and that stability is maintained for at least 6 months.

### Example 5

In this trial, another bacterial strain is tested. Two formulations using Lactobacillus strain bacteria were stabilised and tested for storage stability when stored in vacuum packed aluminium foil bags.

The raw material was freeze dried cells of Lactobacillus obtained from a commercial supplier and received in sealed foil packages stored at 4° C. The freeze dried powder had a water activity of approximately 0.279.

The two formulations were prepared as follows:

### Formulation 5 (F5):

(a) 1 gram of freeze dried powder was mixed with 2 grams of extra virgin olive oil;
(b) 40 μl of prepared antioxidant (mixed tocopherol) was added;
(c) 0.33 gram biopolymer (gellan gum) was added;
(d) 20 grams of oven dried potato starch powder was then added to the bulk in 5 gram increments where the potato starch powder before addition had a water activity (A_{W}) of 0.116.

### Formulation 6 (F6):

(a) 1 gram of freeze dried powder was mixed with 2 grams of extra virgin olive oil;
(b) 40 μl of prepared antioxidant (mixed tocopherol) was added;
(c) 0.17 grams gellan gum and 0.17 grams guar gum was added being the biopolymer;
(d) 20 grams of oven dried rice powder (Paselli BC (1:1)) was then added to the bulk in 5 gram increments where the rice powder before addition had a water activity (A_{W}) of 0.024.

The resulting mixture had a fine crumble texture.

10 gram samples from each formulation were then placed in vacuum packaged foil sachets and stored at 25° C. to test stability over time. When tested, the sachets were destructively sampled and bacterial density enumerated by anaerobic culture on Reinforced Clostridial Agar (RCA). Water activity for the formulation before and after storage was also tested for Formulation F6.

### Results

As shown in Table 5 below, the water activity after mixing and after storage showed little change. Water activity in both cases was extremely low (all less than 0.1).

TABLE 5
Measured Water Activity - Lactobacillus - F6

| | Tested Water Activity | Tested Water Activity | Tested Water Activity | Tested Water Activity | Tested Water Activity |
|---|---|---|---|---|---|
| Formulation | After Mixing (t = 0) | After 1 week | After 2 weeks | After 3 weeks | After 4 weeks |
| F6 | 0.069 | 0.047 | 0.038 | 0.045 | 0.037 |

Stability results shown below in Table 6 showed that there was a less than 1 log loss in bacterial cell counts over a time period of at least 2 months.

TABLE 6
Measured Stability- Lactobacillus - F5, F6

| | Bacterial Count | Bacterial Count | Bacterial Count | Bacterial Count | Bacterial Count | Bacterial Count |
|---|---|---|---|---|---|---|
| Formulation | After Mixing (t = 0) | After 1 week | After 4 weeks | After 8 weeks | After 12 weeks | After 20 weeks |
| Number | (cfu/g) | (cfu/g) | (cfu/g) | (cfu/g) | (cfu/g) | (cfu/g) |
| F5 | 8.47E+08 | 8.93E+08 | 3.89E+08 | 1.20E+09 | 1.62E+09 | 1.38E+09 |
| F6 | 7.51E+08 | 7.08E+08 | 6.40E+08 | 5.49E+08 | 1.17E+09 | 1.31E+09 |

The above trial confirmed that different bacterial strains may be used in the method without altering the improved stabilisation results.

### Example 6

In this trial, another bacterial strain was tested. Two formulations using an alternative Bifidobacterium strain to B. infantis were stabilised and tested for storage stability when stored in vacuum packed aluminium foil bags.

The raw material was freeze dried cells of Bifidobacterium obtained from a commercial supplier and received in sealed foil packages stored at 4° C. The freeze dried powder had a water activity of approximately 0.532.

The two formulations were prepared as follows:

### Formulation 7 (F7):

(a) 1 gram of freeze dried powder was mixed with 2 grams of extra virgin olive oil;
(b) 40 μl of prepared antioxidant (mixed tocopherol) was added;
(c) 0.33 grams biopolymer (gellan gum) was added;
(d) 20 grams of oven dried potato starch powder was then added to the bulk in 5 gram increments where the potato starch powder before addition had a water activity (A_{W}) of 0.116.

### Formulation 8 (F8):

(a) 1 gram of freeze dried powder was mixed with 2 grams of extra virgin olive oil;
(b) 40 μl of prepared antioxidant (mixed tocopherol) was added;
(c) 0.17 grams gellan gum and 0.17 grams guar gum was added being the biopolymer;
(d) 20 grams of oven dried rice powder (Paselli BC (1:1)) was then added to the bulk in 5 gram increments where the rice powder before addition had a water activity (A_{W}) of 0.024.

The resulting mixture had a fine crumble texture.

10 gram samples from each formulation were then placed in vacuum packaged foil sachets and stored at 25° C. to test stability over time. When tested, the sachets were destructively sampled and bacterial density enumerated by anaerobic culture on Reinforced Clostridial Agar (RCA). The water activity was also tested before and after storage.

### Results

As shown in Table 7 below, the water activity after mixing and after storage showed little change. Water activity in both cases was extremely low (all less than 0.1).

TABLE 7
Measured Water Activity - Bifidobacterium - F7, F8

| | Tested Water Activity | Tested Water Activity | Tested Water Activity | Tested Water Activity | Tested Water Activity |
|---|---|---|---|---|---|
| Formulation | After Mixing (t = 0) | After 1 week | After 2 weeks | After 3 weeks | After 4 weeks |
| F7 | 0.069 | 0.055 | 0.052 | 0.048 | 0.050 |
| F8 | 0.074 | 0.037 | 0.041 | 0.044 | 0.033 |

Stability results shown below in Table 8 showed that there was a less than 1 log loss in bacterial cell counts over a time period of at least 2 months.

TABLE 8
Measured Stability - Bifidobacterium - F7, F8

| | Bacterial Count | Bacterial Count | Bacterial Count | Bacterial Count | Bacterial Count | Bacterial Count |
|---|---|---|---|---|---|---|
| Formulation | After Mixing (t = 0) | After 1 week | After 4 weeks | After 8 weeks | After 12 weeks | After 20 weeks |
| Number | (cfu/g) | (cfu/g) | (cfu/g) | (cfu/g) | (cfu/g) | (cfu/g) |
| F7 | 2.09E+09 | 3.34E+09 | 4.62E+09 | 6.92E+09 | 2.15E+09 | 4.82E+09 |
| F8 | 2.02E+09 | 2.92E+09 | 2.29E+09 | 4.38E+09 | 1.61E+09 | 4.80E+09 |

The above trial further confirmed that different bacterial strains may be used in the method without altering the improved stabilisation results.

### Example 7

As noted above, the method works to stabilise biological materials when stored in vacuum sealed environments. In this trial, a freeze dried B. infantis strain was stabilised and then placed into a polystyrene container approximately ⅓ filled with formulation and then sealed without evacuation of oxygen or flushing nitrogen gas. The aim was to determine the effect, if any, that packaging has on storage stability and if vacuum sealing can be avoided during processing.

Freeze dried cells of B. infantis were obtained from a commercial supplier which were delivered in sealed foil packages stored at −20° C. The freeze dried powder had a water activity of approximately 0.4.

The two formulations were prepared as follows:

### Formulation 9 (F9):

(a) 1 gram of freeze dried powder was mixed with 2 grams of extra virgin olive oil;
(b) 40 μl of prepared antioxidant (mixed tocopherol) was added;
(c) 0.17 grams gellan gum and 0.17 grams guar gum was added being the biopolymer;
(d) 20 grams of oven dried rice powder (Paselli BC (1:1)) was then added to the bulk in gram increments where the rice powder before addition had a water activity (A_{W}) of 0.024.

### Formulation 10 (F10):

(a) 1 gram of freeze dried powder was mixed with 2 grams of salad and cooking oil;
(b) 40 μl of prepared antioxidant (mixed tocopherol) was added;
(c) 0.17 grams gellan gum and 0.17 grams guar gum was added being the biopolymer;
(d) 20 grams of oven dried rice powder (Paselli BC (1:1)) was then added to the bulk in 5 gram increments where the rice powder before addition had a water activity (A_{W}) of 0.024.

The final water activities were measured before placing 10 gram samples of each formulation in polystyrene containers filled to approximately ⅓ deep with formulation and then sealed (no vacuum) and stored at 25° C. Bacterial density was measured at intervals enumerated by anaerobic culture on Reinforced Clostridial Agar (RCA) to determine stability. Water activity after storage was also measured.

### Results

As shown in Table 9 below, the water activity after mixing and after storage showed little change. Water activity in both cases was extremely low (all less than 0.1).

TABLE 9
Measured Water Activity - No Vacuum - B. infantis - F9, F10

| | | Tested Water Activity |
|---|---|---|
| Formulation | Tested Water Activity | After Storage (t = 4 |
| Number | After Mixing (t = 0) | weeks) |
| F9 | 0.073 | 0.066 |
| F10 | 0.082 | 0.053 |

Stability results shown below in Table 10 showed that there was a less than 1 log loss in bacterial cell counts over a time period of at least 4 weeks and showing that there is no need to vacuum package as this step has a negligible influence on bacterial stability once the bacteria is stabilised using the method of the present invention.

TABLE 10
Measured Stability - No Vacuum - B. infantis - F9, F10

| | | Bacterial Count |
|---|---|---|
| Formulation | Bacterial Count After | After 4 weeks |
| Number | Mixing (t = 0) (cfu/g) | (cfu/g) |
| F9 | 2.46E+09 | 1.99E+09 |
| F10 | 1.38E+09 | 2.45E+09 |

The above trial shows that vacuum packaging is not essential to maintaining stability.

### Example 8

A separate trial using Formulation 9 of Example 7 was tested over a longer time period to confirm the observed stability noted in Example 7.

### Results

Stability results shown below in Table 11 showed that there was a less than 1 log loss in bacterial cell counts over a time period of at least 6 months showing that vacuum packaging has a minimal influence on bacterial stability once the bacteria is stabilised using the method of the present invention.

TABLE 11
Measured Stability-No Vacuum - B. infantis - Longer Duration - F9

| | Bacterial Count | Bacterial Count | Bacterial Count | Bacterial Count | Bacterial Count |
|---|---|---|---|---|---|
| Formulation | After Mixing (t = 0) | After 1 month | After 2 months | After 4 months | After 6 months |
| Number | (cfu/g) | (cfu/g) | (cfu/g) | (cfu/g) | (cfu/g) |
| F9 | 3.80E+09 | 2.16E+09 | 2.99E+09 | 3.39E+09 | 1.83E+09 |

The above trial shows that vacuum packaging is not essential to maintaining stability.

### Example 9

Formulation 9 (F9) in Example 7 was repeated but with the bacteria substituted with another Bifidobacterium strain to determine the results from using a different bacteria. The new formulation was termed ‘F11’.

### Results

Stability results shown below in Table 12 showed that there was a less than 1 log loss in bacterial cell counts over a time period of at least 12 weeks showing that vacuum packaging has negligible influence on bacterial stability once the bacteria is stabilised using the method of the present invention. The results also show that the same findings apply to different biological materials.

TABLE 12
Measured Stability- No Vacuum - Bifidobacterium -F11

| | Bacterial Count | Bacterial Count | Bacterial Count | Bacterial Count | Bacterial Count | Bacterial Count |
|---|---|---|---|---|---|---|
| Formulation | After Mixing (t = 0) | After 1 week | After 2 weeks | After 4 weeks | After 8 weeks | After 12 weeks |
| Number | (cfu/g) | (cfu/g) | (cfu/g) | (cfu/g) | (cfu/g) | (cfu/g) |
| F11 | 1.73E+09 | 2.36E+09 | 2.22E+09 | 3.01E+09 | 1.09E+09 | 3.47E+09 |

The above trial confirms that vacuum packaging is not essential to maintaining stability even for varying biological materials.

### Example 10

The method of Formulation 9 in Example 7 was repeated but with the bacteria substituted with a Lactobacillus strain to determine the affect if any on stability using a different bacteria (termed formulation 12 or ‘F12 ’). In this Example, bacterial density was enumerated by anaerobic culture on De Man, Rogosa and Sharpe Agar (MRS-Agar)+L-Cysteine-HCl (0.05%).

### Results

Stability results shown below in Table 13 showed that there was a less than 1 log loss in bacterial cell counts over a time period of at least 12 weeks showing that packaging has a negligible influence on bacterial stability once the bacteria is stabilised using the method of the present invention. The results also show that the same findings apply to different biological materials.

TABLE 13
Measured Stability- No Vacuum - Lactobacillus - F12

| | Bacterial Count | Bacterial Count | Bacterial Count | Bacterial Count | Bacterial Count | Bacterial Count |
|---|---|---|---|---|---|---|
| Formulation | After Mixing (t = 0) | After 1 week | After 2 weeks | After 4 weeks | After 8 weeks | After 12 weeks |
| Number | (cfu/g) | (cfu/g) | (cfu/g) | (cfu/g) | (cfu/g) | (cfu/g) |
| F12 | 3.24E+08 | 7.03E+08 | 6.68E+08 | 1.16E+09 | 5.28E+08 | 4.77E+08 |

The above trial confirms that vacuum packaging is not essential to maintaining stability.

### Example 11

The above results show promising stability for sample storage at 25° C. To determine the effects, if any, of humidity, a further trial was completed.

Freeze dried cells of B. infantis were obtained from a commercial supplier which were delivered in sealed foil packages stored at −20° C. The freeze dried powder had a water activity of approximately 0.4.

One formulation was prepared as follows:

### Formulation 13 (F13):

(a) 1 gram of freeze dried powder was mixed with 2 grams of extra virgin olive oil;
(b) 40 μl of prepared antioxidant (mixed tocopherol) was added;
(c) 0.17 grams gellan gum and 0.17 grams guar gum was added being the biopolymer;
(d) 20 grams of oven dried rice powder (Paselli BC (1:1)) was then added to the bulk in 5 gram increments where the rice powder before addition had a water activity (A_{W}) of 0.024.

The final water activities were measured before a sample of formulated bacteria and unformulated bacteria (UF1) were sealed into a dessicator operating at 60% relative humidity and stored in an environment kept at 25° C.

Bacterial density was enumerated at sampling by anaerobic culture on. Reinforced Clostridial Agar (RCA).

### Results

As shown in Table 14, elevated humidity did influence stability but at very different rates. Untreated samples rapidly lost viability with almost all initial viability gone within 3 days. In contrast, the formulated samples showed a significantly greater level of stability with significant numbers of viable bacteria present even after 6 days of humidity exposure.

TABLE 14
Measured Stability - Humidity Influence - B. infantis - F13, UF1

| | Survival Rate | Survival Rate | Survival Rate |
|---|---|---|---|
| | compared to | compared to | compared to |
| | initial readings | initial readings | initial readings |
| | Day 1 | Day 3 | Day 6 |
| Treatment | (%) | (%) | (%) |
| Formulated | 56.45 | 43.00 | 11.76 |
| (F13) | | | |
| Unformulated | 18.99 | 0.08 | 0.00 |
| (UF1) | | | |

The initially low reading for the untreated sample shows the extreme instability of the bacteria when left untreated. The untreated sample showed significant degradation even after one day. The formulated bacteria has a clearly improved stability compared with untreated bacteria even under humid conditions.

The above results also show the importance of achieving a low water activity in order to stabilise the bacteria. Ordinary freeze drying traditionally used to stabilise is insufficient as shown in the above example where the bacteria counts still reduce rapidly even though freeze drying occurred. It should be appreciated that even this result found for humidity degradation is still a significant improvement on the prior art and is useful in product manufacture and handling. For example, short term exposure to humidity of formulated product during further processing should not detrimentally alter viability whereas this is currently a very critical issue.

### Example 12

To determine the effect, if any, of the storage environment, freeze dried cells of Lactobacillus acidophilus were formulated as per Example 1 using gellan and guar gums.

To assess the effect of storage environment, the prepared samples were exposed to controlled humidity environments (20%, 30%, 40% and 50% relative humidity) and temperature (20° C., 30° C., 40° C. and 50° C.) for 2 days, after which time half the samples were left open to the environment and the other half were sealed in foil sachets. In both cases, the open and sealed samples were kept at the same temperature and humidity as during the initial 2 day incubation. After 1 week, the bacterial density per sample was enumerated.

### Results

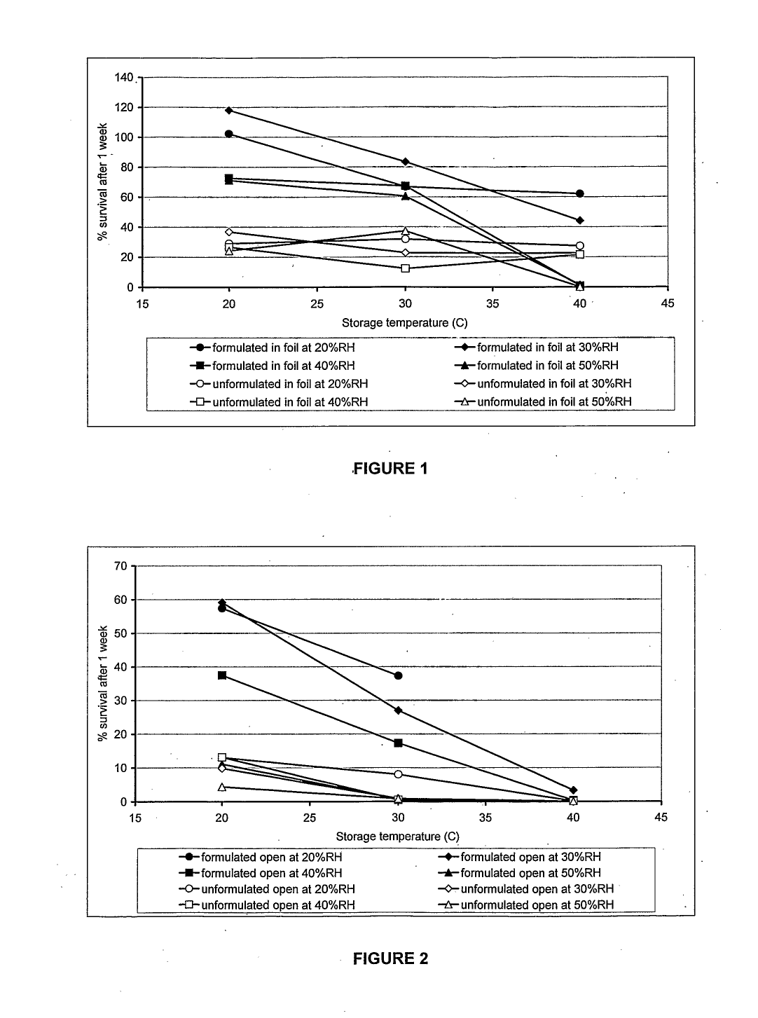

FIGS. 1 and 2 show that for the formulated Lactobacillus acidophilus survival was greater than for the unformulated Lactobacillus acidophilus in open or closed packaging at the storage humidity's and temperatures at which samples were exposed.

This example illustrates the ability of the method to stabilise bacteria in trying conditions even when exposed to the environment. As should be appreciated, by completing the above method, the dried bacteria can be further processed, stored and have a shelf life post unsealing that would not have been contemplated previously, particularly in warm and humid environments.

### Example 13

To illustrate the use of another strain of bacteria, freeze dried cells of Bifidobacterium lactis were formulated as per Example 1 using gellan and guar gums. Samples were compared to unformulated cells by storing at 25° C. and 30° C. and monitoring cell survival.

### Results

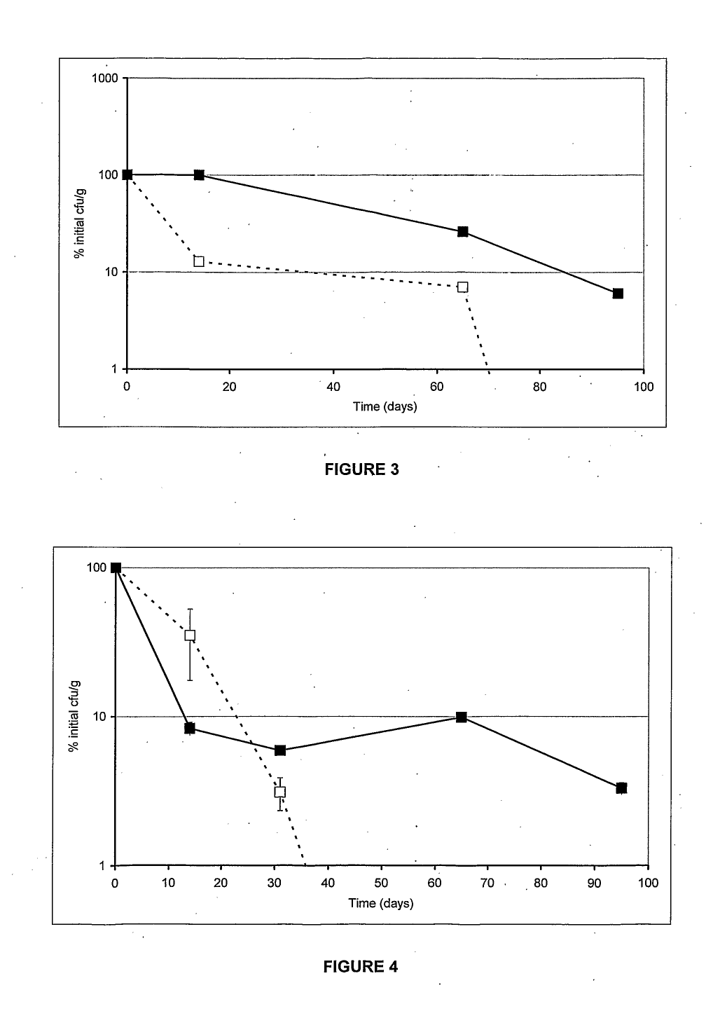

FIGS. 3 and 4 show that the unformulated Bifidobacterium lactis (empty symbols) do not survive as well as those that have been formulated (closed symbols). Like in earlier examples, this example shows that the stabilisation effect is repeatable and various bacteria genus.

### Example 14

To illustrate the use of another strain of bacteria, freeze dried cells of Lactobacillus rhamnosus GG (LGG) strain were formulated as per Example 1 using gellan and guar gums. Samples were compared to unformulated cells by storing at 25° C. and 30° C. and monitoring cell survival.

### Results

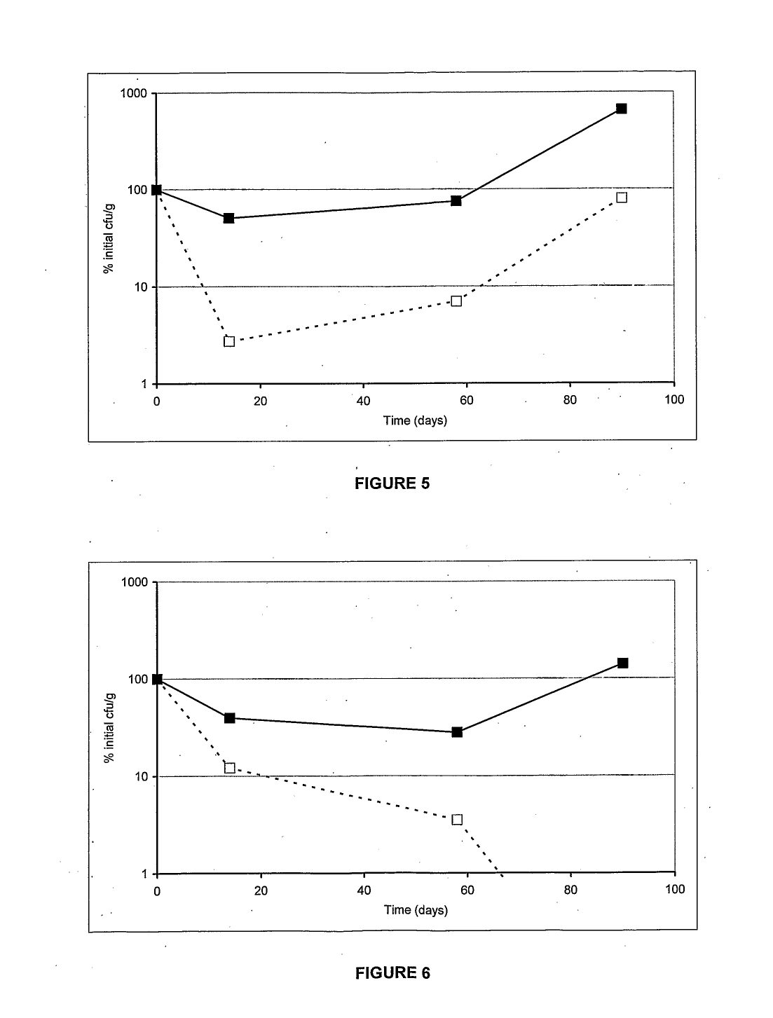

As expected, FIG. 5 and FIG. 6 show that unformulated Lactobacillus GG (empty symbols) does not survive as well as those that have been formulated (closed symbols). This example again shows that the method of the invention is able to be used for a variety of bacteria.

### Example 15

A series of investigations were carried out to establish if some alternate materials might have an intrinsic potential to stabilise probiotic bacteria and therefore confound the results observed.

### Materials

Freeze dried cells of three Lactobacillus species (L. rhamnosus, L. acidophilus, L. casei) were suspended in extra virgin olive oil to a volume of 120 ml. The sample was split into two 3×20 ml samples and stored in open vials at 30° C. and 37° C. at 30% relative humidity.

Control samples were freeze dried powders of the same Lactobacillus species diluted with skim milk powder to 120 grams. The sample was split into two 3×20 gram samples and stored at 30° C. and 37° C. in open vials at 30% relative humidity.

In both cases, 1 gram samples were removed and enumerated for colony forming units at 0, 2 and 4 weeks.

### Results

In all cases, freeze dried bacteria were not stable when suspended in oil or milk powder when stored at 30° C. or 37° C. and 30% relative humidity with losses being unacceptable (2 to 5 log loss) after as little as 2 weeks. Full results are shown in Tables 15 to 17 below.

TABLE 15
Average (n = 3) stability (cfu/g or cfu/mL) of L. rhamnosus freeze
dried powder in oil or skim milk powder at 30° C. and
37° C. and 30% relative humidity

| Formulation | Temperature | Initial | 2 weeks | 4 weeks |
|---|---|---|---|---|
| Oil | 30° C. | 7.35 × 10⁷ | <1.00 × 10³ | 0 |
| Milk powder | 30° C. | 3.00 × 10⁷ | 7.39 × 10⁴ | 1.30 × 10³ |
| Oil | 37° C. | 7.35 × 10⁷ | 6.70 × 10² | 0 |
| Milk powder | 37° C. | 3.00 × 10⁷ | 1.77 × 10⁴ | 5.40 × 10¹ |

TABLE 16
Average (n = 3) stability (cfu/g or cfu/mL) of L. acidophilus freeze
dried powder in oil or skim milk powder at 30° C. and
37° C. and 30% relative humidity

| Formulation | Temperature | Initial | 2 weeks | 4 weeks |
|---|---|---|---|---|
| oil | 30° C. | 4.41 × 10⁴ | <1.00 × 10³ | 0 |
| Milk powder | 30° C. | 1.55 × 10⁶ | 1.83 × 10³ | 0 |
| oil | 37° C. | 4.41 × 10⁴ | <1.00 × 10³ | 0 |
| Milk powder | 37° C. | 1.55 × 10⁶ | 1.65 × 10² | 0 |

TABLE 17
Average (n = 3) stability (cfu/g or cfu/mL) of L. casei freeze dried
powder in oil or skim milk powder at 30° C.
and 37° C. and 30% relative humidity

| Formulation | Temperature | Initial | 2 weeks | 4 weeks |
|---|---|---|---|---|
| oil | 30° C. | 1.66 × 10⁴ | <1.00 × 10³ | 0 |
| Milk powder | 30° C. | 1.30 × 10⁷ | 1.37 × 10³ | 0 |
| oil | 37° C. | 1.66 × 10⁴ | <1.00 × 10³ | 0 |
| Milk powder | 37° C. | 1.30 × 10⁷ | <1.00 × 10³ | 0 |

The results show that there is little intrinsic stability attributable to either oil or milk powder alone and instead, stability in the present invention is likely to be due to a combination of factors.

### Example 16

A detailed description of the technology including a description of the physical changes that occur is provided.

The process begins with the addition of largely transparent and low viscosity oil with a dried biological powder which in this example has a fine granular creamy brown coloured texture. The two components are mixed at a ratio of 1 part dried biological material to between 1 to 4 parts oil

On mixing, a thin brown coloured paste forms.

Biopolymer powder which in this example is an off-white coloured powder is added to the oil and dried material mixture and forms a coarse, friable, damp appearing, and brown coloured powder. The biopolymer powder is added at a rate of a rate of 1 part biological material to between 0.25 to 1 parts biopolymer. At this stage the biological material is considered stable.

A further option is shown. The coarse friable damp appearing powder may then be added to a diluent such as lactose which in this example is a white powder. The resulting mixture is a flowable powder produced after blending which is a granular off white coloured and dry looking.

The resulting flowable powder is stable as noted above and has the advantage of being easy to handle which may be an advantage for further processing.

In another embodiment, the coarse friable damp appearing powder may be coated onto a substrate such as a bran flake resulting in a coated flake.

### Example 17

In this example samples were tested for physical stability.

Samples were prepared as per Example 12 and stored at 20%, 30%, 40% or 50% relative humidity and 20° C., 30° C., 40° C. or 50° C. in open containers. Post storage the unformulated material showed dramatic discolouration and caking turning to a yellow brown colour. By contrast, the formulated samples show no change with the colour remaining an off white and the material retaining good flow characteristics.

### Example 18

In this example, the use of a variety of gums was tested to confirm the inventors understanding that a wide variety of gums may be used in the context of the present invention.

The experiment was completed by preparing six types of formulation either using single gum or two gums as described in the results table below. Each of the formulations were replicated (n=2). In more detail, the formulations were prepared by the steps of:
(a) Mixing freeze dried B. infantis (1 gram) with extra virgin olive oil (2 grams).
(b) Adding antioxidant (mixed tocopherol, 40 μl).
(c) Adding the biopolymer gum as a powder either as a single (0.34 gram) or combination of two (0.17 gram each) as described in the table given below.
(d) Adding 20 grams of desiccation agent being a 1:1 ratio mixture of rice powder and paselli BC pre-conditioned at 80° C. for 24 hrs.
(e) Vacuum packing the resulting formulation in foil bags and storing the bags and formulations at 25° C.
(f) Testing the viable cell counts and water activity for each of the formulations at t=0, 1 week and 2 weeks.

The results measured are as shown in Table 18 below.

TABLE 18
Stability (cfu/g) and water activity of B. infantis freeze
dried powder in oil at 25° C. using different biopolymers

| | Viable cell counts (cfu/g) | | | Water activity (a_{w}) | | |
|---|---|---|---|---|---|---|
| Formulation | T0 | T1 wk | T2 wk | T0 | T1 wk | T2 wk |
| Xanthan 1 | 2.03E+09 | 1.69E+09 | 2.94E+09 | 0.062 | 0.047 | 0.061 |
| Xanthan 2 | 3.12E+09 | 1.48E+09 | 2.20E+09 | 0.060 | 0.042 | 0.041 |
| Locust Bean 1 | 3.36E+09 | 1.60E+09 | 3.47E+09 | 0.080 | 0.049 | 0.058 |
| Locust Bean 2 | 3.14E+09 | 2.71E+09 | 3.59E+09 | 0.115 | 0.042 | 0.036 |
| Xanthan + Locust | 2.10E+09 | 1.89E+09 | 7.21E+09 | 0.080 | 0.04 | 0.044 |
| Bean 1 | | | | | | |
| Xanthan + Locust | 2.73E+09 | 2.56E+09 | 2.79E+09 | 0.046 | 0.042 | 0.041 |
| Bean 2 | | | | | | |
| Gellan 1 | 2.23E+09 | 4.00E+09 | 5.12E+09 | 0.064 | 0.039 | 0.037 |
| Gellan 2 | 2.82E+09 | 2.90E+09 | 5.85E+09 | 0.060 | 0.048 | 0.035 |
| Guar 1 | 2.89E+09 | 2.54E+09 | 5.04E+09 | 0.052 | 0.040 | 0.048 |
| Guar 1 | 3.62E+09 | 3.14E+09 | 6.31E+09 | 0.043 | 0.052 | 0.052 |
| Gellan + Guar 1 | 3.51E+09 | 2.23E+09 | 2.78E+09 | 0.096 | 0.046 | 0.039 |
| Gellan + Guar 2 | 2.51E+09 | 3.12E+09 | 3.11E+09 | 0.049 | 0.053 | 0.043 |

The above results show that other biopolymers including xanthan gum and locust bean gum may be used with similar results to that observed for gellan and guar gums.

### Example 19

A further example is now provided illustrating stability at 30° C. and to determine the influence if any of the ratio of oil to biopolymer on stability.

Stabilised powders were produced by:
(a) Mixing 2 grams of freeze dried Lactobacillus acidophilus cells with either 4 grams or 6 grams of olive oil followed by the addition of 804 vitamin E.
(b) Weigh out 0.34 grams of gellan gum and 0.34 grams of guar gum and mix together or weight out 0.68 grams gellan gum or weight out 0.68 grams guar gum.
(c) Combine the gums with the freeze dried cell oil mixture
(d) Add 40 grams of rice powder and potato starch mixed in a 1:1 ratio.
(e) Dry the resulting mixture at 80° C. for 24 hours or until equilibrated to a water activity of 0.3 over a saturated MgCl₂ solution at 30° C.
(f) Store over a saturated MgCl₂ solution for 6 days prior to packaging 5 gram samples in vacuum sealed foil.
(g) Store at 30° C. and monitor cfu/g monthly.

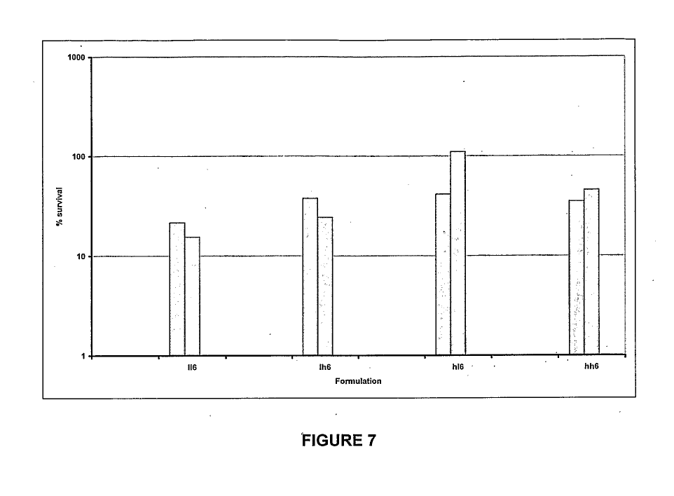

As shown in FIG. 7, survival (%) at 30° C. was comparable to results observed for 25° C. as described above. In addition, a slight improvement was observed for formulations that included extra oil.

### Example 20

Examples are now provided showing uses of the stabilized freeze dried powder of the present invention.

Firstly, a base stabilised powder containing probiotic material is produced by:
(a) Mix 2 grams of freeze dried probiotic material with 4 grams of olive oil followed by 80 μL vitamin E.
(b) Weigh out 0.34 grams of gellan gum and 0.34 grams of guar gum. Mix the gums and this mixture with the freeze dried bacteria oil mixture of step (a).
(c) Add 40 grams of rice powder and potato starch mixed in a 1:1 ratio.

Probiotic containing formulations may then be produced as follows:

### Milk Powder Containing Probiotic

| | | |
|---|---|---|
| Stabilized freeze dried probiotic powder | 10 | g |
| Milk powder | to 100 | g |

### Infant Formula Containing Probiotic

| | | |
|---|---|---|
| Stabilized freeze dried probiotic powder | 5 | g |
| Infant formula | to 100 | g |

### Probiotic Powder for Dogs

| | | |
|---|---|---|
| Vitamin A | 5000 | IU |
| Vitamin C | 4 | g |
| Vitamin E | 800 | IU |
| Vitamin B complex | 0.1 | g |
| Bromelain | 0.1 | g |
| Glutamine | 1.5 | g |
| Arginine | 1 | g |
| Selenium | 75 | mcg |
| Pancreatin | 0.01 | g |
| Stabilized freeze dried probiotic powder | 9 | g |
| Starch | to 25 | g |

The above examples show that there is provided a method of stabilising a dried biological material, particularly dried bacteria such that the stability is remarkably steady for extended periods of time when stored in a dry environment and even when subjected to humidity, the method greatly enhances the stability as well. Even when processed, in unsealed ambient conditions the stability remains, therefore providing a marked improvement over art methods requiring special handling characteristics.

Aspects of the present invention have been described by way of example only and it should be appreciated that modifications and additions may be made thereto without departing from the scope thereof as defined in the appended claims.

1-55. (canceled)

56. A storage stable composition comprising
(1) a biological material comprising at least one microorganism,
(2) an oil coat on the biological material, and
(3) a coat comprising a biopolymer gum and at least one desiccant substance on the oil-coated biological material,
wherein
(a) the composition has a water activity of less than 0.7; and
(b) the viability of the biological material in the composition does not reduce by more than 2 log when stored for at least 1 month at 25° C.

57. The composition as claimed in claim 56 wherein the biological material in the composition has a less than 50% loss in viability when stored at 60% relative humidity and at 25° C. for a time period of at least 3 days.

58. The composition as claimed in claim 56 wherein the biological material contains at least one bacteria or fungi, or, wherein the bacteria is an obligate anaerobe or a probiotic bacteria of a genus selected from the group consisting of Lactobacillus, Bifidobacterium, Streptococcus (Enterococcus), and combinations thereof.

59. The composition as claimed in claim 56 wherein the biological material is dried biological material, or the desiccant substance has a water activity of less than 0.4, or the biological material is dried biological material and the desiccant substance has a water activity of less than 0.4.

60. The composition as claimed in claim 56 wherein the oil is edible oil, vegetable oil or is an oil selected from olive oil, canola oil, sunflower seed oil, hydrolyzed oils, and combinations thereof.

61. The composition as claimed in claim 56 wherein the ratio of dried biological material to oil is between approximately 1:10 to 10:1.

62. The composition as claimed in claim 56 wherein the biopolymer gum is selected from the group consisting of: agar, alginate, cassia, dammar, pectin, beta-glucan, glucomannan, mastic, chicle, psyllium, spruce, gellan, guar, locust bean, xanthan, and combinations thereof.

63. The composition as claimed in claim 56 wherein the ratio of biological material to biopolymer gum is approximately 1 part biological material to 0.25 or 1 part biopolymer.

64. The composition as claimed in claim 56 wherein the desiccant substance is selected from the group consisting of: rice powder, corn starch powder, potato starch powder, lactose, sucrose, glucose, mannitol, sorbitol, calcium carbonate, silicone dioxide, calcium phosphate, celluloses, polyethylene glycol, and combinations thereof.

65. The composition as claimed in claim 56 wherein the composition is in the form of a powder or a crumble.

66. A food, food ingredient or nutraceutical product including the storage stable composition as claimed in any one of claims 56 to 67 or a bran flake coated in the composition as claimed in any one of claims 56 to 67.

67. A method of producing a storage stable composition including a stabilised biological material by the steps of:
(a) providing a dried biological material comprising at least one microorganism;
(b) coating the dried biological material with at least one oil to form a oil coated material;
(c) coating the oil coated material with at least one biopolymer gum powder and at least one desiccant substance,
wherein
(i) the composition has a water activity of less than 0.7; and
(ii) the viability of the biological material in the composition does not reduce by more than 2 log when stored for at least 1 month at 25° C.

68. The method as claimed in claim 67 wherein the dried biological material is pre-processed by drying.

69. The method as claimed in claim 67 wherein the dried biological material is a powder with a particle size of less than approximately 2 mm or less than 200 μm, or the biopolymer gum is a powder with a particle size of less than 2 mm, or the dried biological material is a powder with a particle size of less than approximately 2 mm or less than 200 μm and the biopolymer gum is a powder with a particle size of less than 2 mm.

70. The method as claimed in claim 67 wherein the desiccants are added at a rate of 1 to 10 gram desiccant per stage per 1 gram of dried cells selected in step (a) or wherein a total of up to 20 grams of desiccants are used per 1 gram of dried cells selected in step (a).

71. The method as claimed in claim 67 wherein the desiccants are added in multiple stages to the composition of step (c) or wherein sufficient desiccant is added to form a rough crumble.

72. The method as claimed in claim 67 wherein the composition produced is further processed into forms selected from the group consisting of: extrusions, tablets, granules, prills, and pellets.

73. The method as claimed in claim 67 wherein the difference in cell count before and after processing is less than 2 log loss.

74. The method as claimed in claim 67 wherein the composition produced is coated onto a substrate.

75. A product, food product, food ingredient, or nutraceutical product produced by the process as claimed in claim 67 or a bran flake coated in the composition produced by the process as claimed in claim 67.
